SEQUENCE LISTING (53 sequences)

SEQ ID NO: 1 - DNA, 30 bp, artificial sequence
  Probe for Cp. psittaci genotype A
ctaccgatcttccaacgcaacttcctaacg

SEQ ID NO: 2 - DNA, 33 bp, artificial sequence
  Probe for Cp. psittaci genotype B
tctaccgatcttccaatgcaacttcctaacgta

SEQ ID NO: 3 - DNA, 31 bp, artificial sequence
  Probe for Cp. psittaci genotype C
tctgctgttatgaacttgaccacatggaacc

SEQ ID NO: 4 - DNA, 24 bp, artificial sequence
  Probe for Cp. psittaci genotype D
aggaaaggccacaactgtcgacgg

SEQ ID NO: 5 - DNA, 36 bp, artificial sequence
  Probe for Cp. psittaci genotype E
tactttgcccaataatggtggtaaggatgttctatc

SEQ ID NO: 6 - DNA, 27 bp, artificial sequence
  Probe for Cp. psittaci genotype F
catcgctcaacctaaattagccgctgc

SEQ ID NO: 7 - DNA, 34 bp, artificial sequence
  competitor probe
tctaccgatccttccaacgcaacttcctaacgta

SEQ ID NO: 8 - DNA, 30 bp, artificial sequence
  competitor probe
ctaccgatcttccaatgcaacttcctaacg

SEQ ID NO: 9 - DNA, 36 bp, artificial sequence
  competitor probe
tgctttgcccaataatgctggtaaggatgttctatc

SEQ ID NO: 10 - DNA, 33 bp, artificial sequence
  competitor probe
tctaccgagcttccaatgcaacttcctaacgta

SEQ ID NO: 11 - DNA, 36 bp, artificial sequence
  competitor probe
tgctttgcccaataatagtggtaaggatgttctatc

SEQ ID NO: 12 - DNA, 21 bp, artificial sequence
  forward primer genotype A
ggttttcagctgcaagctcaa

SEQ ID NO: 13 - DNA, 21 bp, artificial sequence
  reverse primer genotype A
ccacaacaccttgggtaatgc

SEQ ID NO: 14 - DNA, 27 bp, artificial sequence
  forward primer genotype B
baatagggttttcagctaccaactcaa

SEQ ID NO: 15 - DNA, 21 bp, artificial sequence
  reverse primer genotype B
ccacaacaccttgggtaatgc

SEQ ID NO: 16 - DNA, 21 bp, artificial sequence
  forward primer genotype C
gcatcgctcaacctaaattgg

SEQ ID NO: 17 - DNA, 21 bp, artificial sequence
  reverse primer genotype C
attgtggcttcccctaaaagg

SEQ ID NO: 18 - DNA, 23 bp, artificial sequence
  forward primer genotype D
aaccacttggaacccaacacttt

SEQ ID NO: 19 - DNA, 28 bp, artificial sequence
  reverse primer genotype D
cgaagcaagttgtaagaagtcagagtaa

SEQ ID NO: 20 - DNA, 22 bp, artificial sequence
  forward primer genotype E
ccaagccttctaggatcaagga

SEQ ID NO: 21 - DNA, 22 bp, artificial sequence
  reverse primer genotype E
cgaagcaatttgcaagacatca

SEQ ID NO: 22 - DNA, 25 bp, artificial sequence
  forward primer genotype F
gcaacttttgatgctgactctatcc

SEQ ID NO: 23 - DNA, 24 bp, artificial sequence
  reverse primer genotype F
gttccatgtggtcaagttcaaaac

SEQ ID NO: 24 - DNA, 22 bp, artificial sequence
  Probe for Cp. psittaci genotype EB
ccaagccttctaggatcaacca

SEQ ID NO: 25 - DNA, 20 bp, artificial sequence
  forward primer genotype EB
tgctttgcccaataatgctg

SEQ ID NO: 26 - DNA, 26 bp, artificial sequence
  reverse primer genotype EB
aaggatgttctatctgatgtcttgca

SEQ ID NO: 27 - DNA, 101 bp, Chlamydophila psittaci
gttgggttaatagggttttcagctgcaagctcaatctctaccgatcttccaacgcaacttcctaacgtaggcattacccaaggtgttgtggaattttatac

SEQ ID NO: 28 - DNA, 101 bp, Chlamydophila psittaci
gttgggttaatagggttttcagctaccaactcaacctctaccgatcttccaatgcaacttcctaacgtaggcattacccaaggtgttgtggaattttatac

SEQ ID NO: 29 - DNA, 89 bp, Chlamydophila psittaci
gttggtttgattggtgttaaaggaagctccttaacaaatgaccaacttcccaacgtagccatcactcaaggcgttgttgagttttacac

SEQ ID NO: 30 - DNA, 86 bp, Chlamydophila psittaci
gttggtttgattggtcttaaaggaactgatttcaataatcaacttccaaacgtagccatcacccaaggcgttgttgagttttacac

SEQ ID NO: 31 - DNA, 101 bp, Chlamydophila psittaci
gttgggttaatagggttttcagctaccagctcaacctctaccgagcttccaatgcaacttcctaacgtaggcattacccaaggtgttgtggaattttatac

SEQ ID NO: 32 - DNA, 89 bp, Chlamydophila psittaci
gttggtttgattggtgttaaaggaacctccgtagcagctgatcaacttccaaacgtaggcatcactcaaggtattgttgagttttacac

SEQ ID NO: 33 - DNA, 101 bp, Chlamydophila psittaci
atactatccgcattgctcaacctaaattaaaatcggagattcttaacattactacatggaacccaagccttataggatcaaccactgctttgcccaataat

SEQ ID NO: 34 - DNA, 101 bp, Chlamydophila psittaci
atactatccgcattgctcaacctaaattaaaatcggagattcttaacattactacatggaacccaagccttctaggatcaaccactgctttgcccaataat

SEQ ID NO: 35 - DNA, 95 bp, Chlamydophila psittaci
acactatccgcatcgctcaacctaaattggcctctgctgttatgaacttgaccacatggaacccaacccttttaggggaagccacaatgcttgat

SEQ ID NO: 36 - DNA, 95 bp, Chlamydophila psittaci
acactatccgcattgctcagcctaaattagccactgctgttttagatttaaccacttggaacccaacacttttaggaaaggccacaactgtcgac

SEQ ID NO: 37 - DNA, 101 bp, Chlamydophila psittaci
atactatccgcattgctcaacctaaattaaaatcggagattcttaacattactacatggaacccaagccttctaggatcaaccactactttgcccaataat

SEQ ID NO: 38 - DNA, 95 bp, Chlamydophila psittaci
actctatccgcatcgctcaacctaaattagccgctgctgttttgaacttgaccacatggaacccaactcttttaggggaagctacagctttagat

SEQ ID NO: 39 - DNA, 101 bp, Chlamydophila psittaci
acattactacatggaacccaagccttataggatcaaccactgctttgcccaataatagtggtaaggatgttctatctgatgtcttgcaaattgcttcgatt

SEQ ID NO: 40 - DNA, 101 bp, Chlamydophila psittaci
acattactacatggaacccaagccttctaggatcaaccactgctttgcccaataatagtggtaaggatgttctatctgatgtcttgcaaattgcttcgatt

SEQ ID NO: 41 - DNA, 92 bp, Chlamydophila psittaci
acttgaccacatggaacccaacccttttaggggaagccacaatgcttgatacttccaataaattcagtgacttcttacaaatcgcttcgatt

SEQ ID NO: 42 - DNA, 92 bp, Chlamydophila psittaci
atttaaccacttggaacccaacacttttaggaaaggccacaactgtcgacggtaccaatacttactctgacttcttacaacttgcttcgatt

SEQ ID NO: 43 - DNA, 100 bp, Chlamydophila psittaci
acattactacatggaacccaagccttctaggatcaaccactactttgcccaataatggtggtaaggatgttctatctgatgtcttgcaaattgcttcgat

SEQ ID NO: 44 - DNA, 97 bp, Chlamydophila psittaci
acttgaccacatggaacccaactcttttaggggaagctacagctttagatgctagcaacaaattctgcgacttcttacaaatcgcttcgattcagat

SEQ ID NO: 45 - DNA, 101 bp, Chlamydophila psittaci
taaactggtcaagagcaacttttgatgctgatactatccgcattgctcaacctaaattaaaatcggagattcttaacattactacatggaacccaagcctt

SEQ ID NO: 46 - DNA, 101 bp, Chlamydophila psittaci; identical to SEQ ID NO: 45

SEQ ID NO: 47 - DNA, 101 bp, Chlamydophila psittaci
taaactggtcacgagcaacttttgatgccgacactatccgcatcgctcaacctaaattggcctctgctgttatgaacttgaccacatggaacccaaccctt

SEQ ID NO: 48 - DNA, 101 bp, Chlamydophila psittaci
taaactggtcaagagcaacttttgatgctgacactatccgcattgctcagcctaaattagccactgctgttttagatttaaccacttggaacccaacactt

SEQ ID NO: 49 - DNA, 101 bp, Chlamydophila psittaci; identical to SEQ ID NO: 45

SEQ ID NO: 50 - DNA, 101 bp, Chlamydophila psittaci
taaactggtcaagagcaacttttgatgctgactctatccgcatcgctcaacctaaattagccgctgctgttttgaacttgaccacatggaacccaactctt

SEQ ID NO: 51 - DNA, 101 bp, Chlamydophila psittaci
acattactacatggaacccaagccttctaggatcaaccactgctttgcccaataatgctggtaaggatgttctatctgatgtcttgcaaattgcttcgatt

SEQ ID NO: 52 - DNA, 21 bp, Chlamydophila psittaci
atgaaaaaactcttgaaatcg

SEQ ID NO: 53 - DNA, 21 bp, Chlamydophila psittaci
acaagcttttctagacttcat

(19) United States
(12) Patent Application Publication
(10) Pub. No.: US 2006/0008828 A1
(43) Pub. Date: Jan. 12, 2006

(54) Genotype specific detection of Chlamydophila psittaci

(76) Inventor: Daisy Vanrompay, Balegem (BE)

Correspondence Address: CLARK & ELBING LLP, 101 FEDERAL STREET, BOSTON, MA, 02110, US

(21) Appl. No.: 11/171,113

(22) Filed: Jun. 30, 2005

Related U.S. Application Data
(60) Provisional application No. 60/584,725, filed on Jun. 30, 2004.

(51) Int. Cl. C12Q 1/68

(52) U.S. Cl. 435/6

(57) ABSTRACT

The present invention describes novel methods for the specific detection and identification of Chlamydophila psittaci genotypes. According to one embodiment the method makes use of quantitative PCR with internal probes and optionally competitor probes which increase specificity. The invention also describes a strain of Cp. psittaci with a novel genotype EB and methods to distinguish said novel genotype from previously identified genotypes.

### CROSS-REFERENCE TO RELATED APPLICATIONS

This application claims benefit of U.S. provisional patent application Ser. No. 60/584,725, filed Jun. 30, 2004, the disclosure of which is hereby incorporated by reference.

### FIELD OF THE INVENTION

The present invention relates to the qualitative and quantitative detection of genotypes of Chlamydiaceae as well as to the detection and diagnosis of bacterial infections in mammals, including humans and birds. The invention further relates to the detection of a novel strain of an infectious bacterium.

### BACKGROUND OF THE INVENTION

Bacteria in the family of the Chlamydiaceae are obligate intracellular parasites of eukaryotic cells. In animals, Chlamydophilae are capable of inducing a broad spectrum of symptoms like enteritis, urogenital infection, abortion, pneumonia, polyarthiritis, polyserositis, encephalitis and mastitis. Chlamydophila (Cp.) psittaci (formerly Chlamydia psittaci) causes respiratory diseases in birds and psittacosis or parrot-fever in man. Until now detection of Cp. psittaci in avian samples is routinely performed by direct visualisation of the organisms using cytological stainings, by isolation in cell culture or specific pathogen-free embryonated eggs, by detection of Cp. psittaci antigens or by serologic tests measuring antibodies. Cytological stainings have poor sensitivity and specificity and can only be used as a rapid preliminary investigation method. The main disadvantage of isolation is the need for viable bacteria. This means special requirements for collection and storage of samples, requirements that cannot always be fulfilled when collecting field samples. In addition, isolation is time-consuming and costly and can only be performed in laboratories with a specific biosafety level since Cp. psittaci is a zoonotic agent which spreads by aerosol. The current rapid antigen-detection methods are not recommended for demonstrating Cp. psittaci in individual birds because of shortcomings in either sensitivity or specificity.

Serology is not particularly useful in diagnosing an active Cp. psittaci infection in birds because of the high prevalence of this infection in birds and the long-term (up to several months) persistence of anti-Cp. psittaci antibodies. In addition, antibody detection based on using whole organisms, LPS (LipoPolySaccharides) or outer membrane fractions can generate false positives due to the presence of antibodies cross reactive to the Cp. psittaci LPS or heat shock proteins. Importantly, current Cp. psittaci antibody detection tests cannot be used for demonstrating a Cp. psittaci infection in man, as humans can also become infected with other members of the Chlamydiaceae as Chlamydia trachoinatis, Chlamydophila pneumonieae (formerly Chlamydia pneumoniae) and Chlamydophila abortus (formerly psittaci serotype 1) which can cause false-positive results. Diagnosis of infection with Cp. psittaci has been difficult and cumbersome. Until now, detection of Cp. psittaci in avian samples is done with serological tests, providing, as indicated above, only retrospective information.

Cp. psittaci has been classified into six avian serovars (A to F) using a panel of serovar-specific monoclonal antibodies against the Major Outer Membrane Protein (MOMP). The MOMP is encoded by the OmpA gene and OmpA restriction fragment length polymorphism (RFLP) analysis reveals six corresponding genotypes. Until now, genotype A, C and D are the most common genotypes associated with human psittacosis. While RFLP analysis of the ompA gene encoding the MOMP is allows specific detection of the Cp. psittaci genotypes, restriction patterns in RFLP are sometimes difficult to analyse, and ompA amplification cannot always be carried out directly on clinical samples. Moreover, this method requires the amplification of the entire 1200 bp OmpA gene which often fails when a limited amount of DNA is available. Indirect micro-immunofluorescence (IMIF) with monoclonal antibodies always requires culturing, and is therefore expensive and labour-intensive and is definitely less sensitive then genotyping by means of RFLP or whole ompA sequence analysis. Besides the interspecies diagnosis problems in the serological assays and the intraspecies difficulties when dealing with mixed infections in RFLP or serotyping, these tests all have the problem that they do not provide information about the actual number of infectious particles in the specimen, making it also difficult or impossible to follow up a treatment or to track down the origin of an infection. The present overview illustrates a need for a specific diagnostic test for determining the genotype of Cp. psittaci in birds and mammals including man. Such a test should be rapid and sensitive.

### SUMMARY OF THE INVENTION

In a first aspect, the invention relates to an ex vivo or in vitro method for the identification of the presence of one or more genotypes of Cp. psittaci in a sample. Thus the present invention provides a method for the determination of the presence of Cp. psittaci in a sample as well as a method to specifically identify amongst the different Cp. psittaci genotypes, which genotype is present in the sample, thus allowing the determination of the actively infecting agent, even when the samples is taken from an animal or human subject which has previously been infected with Cp. psittaci.

One specific embodiment of the invention relates to a method for detecting a novel genotype of Cp. psittaci, referred to as genotype EB. Further embodiments of the invention relate to methods for detecting and identifying the presence of the genotypes A, B, C, D, E, and F.

According to a further specific embodiment the ex vivo or in vitro method for the detection and/or identification of the presence of DNA of a genotype of Cp. psittaci in a sample comprises the steps of (a) incubating the sample with a first oligonucleotide which is capable of specifically hybridising to DNA of a genotype of Cp. psittaci, and, (b) determining the binding of the first oligonucleotide to DNA within the sample, which binding is indicative of the presence of DNA of a genotype of Cp. psittaci in that sample. According to specific embodiments of the invention the detection and/or identification is performed using a first nucleotide is comprising a sequence of at least 15 nucleotides of the OmpA gene of one of the Cp. psittaci genotypes, more specifically, comprising a sequence of at least 15 nucleotides within the region from about nucleotide 450 to about nucleotide 600 or from about nucleotide 900 to about 1100 of the OmpA sequence corresponding to GB accession AF269281, or a sequence being essentially identical to a sequence of 15 nucleotides within the OmpA gene, more particularly within these regions of the OmpA gene. Most particular embodiments of the invention encompass methods wherein the first genotype-specific oligonucleotide is selected from the group consisting of sequence corresponding to SEQ ID NO: 1 for genotype A, sequence corresponding SEQ ID NO: 2, for genotype B, sequence corresponding SEQ ID NO: 3 for genotype C, sequence corresponding SEQ ID NO: 4, for genotype D, sequence corresponding SEQ ID NO: 5, for genotype E, sequence corresponding SEQ ID NO: 6, for genotype F and sequence corresponding SEQ ID NO: 25, for genotype EB or a sequence essentially identical thereto capable of hybridising specifically to the respective genotype. Such an oligonucleotide can be labeled e.g. with a chromophoric group at its 5′ and with a quencher group at its 3′ end.

A particular embodiment of the invention relates to the identification of a particular genotype of Cp. psittaci in a sample. It is further envisaged that in alternative embodiments the probes of the present invention can be combined for the simultaneous detection of more than one genotype of Cp. psittaci in a sample.

A further aspect of the invention relates to an ex vivo or in vitro method for the identification of the presence of one or more (first) genotypes of Cp. psittaci in a sample as described above, wherein the specificity of the detection is further improved by the use of a second oligonucleotide which prevents non-specific hybridisation of the first oligonucleotide to the DNA of another genotype of Cp. psittaci. Thus, according to this embodiment of the invention, the sample is incubated with at least one second oligonucleotide in addition to the first oligonucleotide being capable of hybridising specifically to the DNA of a first Cp. psittaci genotype, whereby the second oligonucleotide is a competitor for the hybridisation of this first oligonucleotide to DNA of another genotype of Cp. psittaci. According to particular embodiments of this aspect of the invention the first and second oligonucleotide are selected from the group consisting of (a) a second oligonucleotide comprising the sequence of SEQ ID NO: 8, and a first oligonucleotide comprising the sequence of SEQ ID NO: 1, (b) a second oligonucleotide comprising the sequence of SEQ ID NO: 7, and a first oligonucleotide comprising the sequence of SEQ ID NO: 2; (c) a second oligonucleotide comprising the sequence of SEQ ID NO: 10, and a first oligonucleotide comprising the sequence of SEQ ID NO: 2, (d) a second oligonucleotide comprising the sequence of SEQ ID NO: 9, and a first oligonucleotide-comprising the sequence of SEQ ID NO: 5, and (e) a second oligonucleotide comprising the sequence of SEQ ID NO: 1, and a first oligonucleotide comprising the sequence of SEQ ID NO: 5.

According to a particular embodiment of the method described in both the first and the second aspect of the present invention, the binding of the first oligonucleotide is determined by PCR amplification with a forward and a reverse primer. More particularly the forward and reverse primer are located about 1 to 100 bp 3′ and 5′ from the first oligonucleotide. Specific embodiments of the primers for use in detection of the first oligonucleotide in the context of the present invention are selected from group consisting of (a) primers comprising the sequence of SEQ ID NO: 12 and SEQ ID NO: 13, when the first oligonucleotide comprises the sequence of SEQ ID NO: 1; (b) primers comprising the sequence of SEQ ID NO: 14 and SEQ ID NO: 15 when the first oligonucleotide comprises the sequence of SEQ ID NO: 2; (c) primers comprising the sequence of SEQ ID NO: 16 and SEQ ID NO: 17 when the first oligonucleotide comprises the sequence of SEQ ID NO: 3; (d) primers comprising the sequence of SEQ ID NO: 18 and SEQ ID NO: 19 when the first oligonucleotide comprises the sequence of SEQ ID NO: 4; (e) primers comprising the sequence of SEQ ID NO: 20 and SEQ ID NO: 21 when the first oligonucleotide comprises the sequence of SEQ ID NO: 5; (f) primers comprising the sequence of SEQ ID NO: 22 and SEQ ID NO: 23 when the first oligonucleotide comprises the sequence of SEQ ID NO: 6; (g) primers comprising the sequence of SEQ ID NO: 25 and SEQ ID NO: 26 when the first oligonucleotide comprises the sequence of SEQ ID NO: 24; or primers which have a sequence essentially identical to the above primers for PCR amplification.

Specific embodiments of the method according to both the first and the second aspect of the present invention are methods used for the detection and/or identification of a Cp. psittaci genotype in birds, most particularly for the detection in birds which are in a stage of development in which the maternal immunity disappears. One particular embodiment of the invention is a method for detecting and/or identifying an infection with Cp. psittaci in a duck of about 6 weeks after hatching.

Specific applications of the described embodiments of the method according to both the first and the second aspect of the present invention are the detection and/or identification of a Cp. psittaci infection in a sample in order to determine the efficacy of a treatment against a a Cp. psittaci infection. Thus the present invention further relate to methods for determining the efficacy of treatment of a a Cp. psittaci infection comprising the method steps described above.

Yet another aspect of the present invention relate to diagnostic kits for the detection and/or identification of a Cp. psittaci genotype comprising one or more oligonucleotides capable of hybridizing specifically to a sequence within the DNA of a genotype of Cp. psittaci. Particular embodiments of the diagnostic kit of the invention relate to kits wherein the one or more oligonucleotides are capable of hybridizing specifically to a sequence within the ompA gene of Cp. psittaci. Further specific embodiments relate to kits wherein the one or more oligonucleotides are capable of hybridizing specifically to a sequence within the region from about nucleotide 450 to about nucleotide 600 or from about nucleotide 900 to about 1100 of the OmpA gene sequence corresponding to GB accession AF269281. Particular examples of the diagnostic kit of the invention relate to diagnostic kits for the identification of one or more of the genotypes selected from the group consisting of A, B, C, D, E, F and/or EB, whereby the EB genotype is a novel genotype of Cp. psittaci identified herein. Most particular embodiments of the diagnostic kits of the present invention relate to kits comprising one or more of the oligonucleotides selected from the group consisting of:
genotype-specific oligonucleotides: SEQ ID NO: 1, SEQ ID NO: 2, SEQ ID NO: 3, SEQ ID NO: 4, SEQ ID NO: 5, SEQ ID NO: 6 and 24; and
genotype-specific primers: SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 25 and SEQ ID NO: 26

It will however be understood by the skilled person that genotype-specific oligonucleotides and genotype-specific primers essentially identical to the oligonucleotides and primers described therein can equally be applied in the context of the present invention. Further particular embodiments of the diagnostic kits of the present invention relate to kits comprising two oligonucleotides selected from the genotype-specific oligonucleotides SEQ ID NO: 1, SEQ ID NO: 2, SEQ ID NO: 3, SEQ ID NO: 4, SEQ ID NO: 5, SEQ ID NO: 6 and 24.

Yet a further aspect of the present invention relates to a novel strain of a Cp. psittaci bacterium, designated as Cp. psittaci genotype EB which is characterized in that it comprises the OmpA sequence depicted in SEQ ID NO: 51.

Yet another aspect of the present invention relates to a method of generating oligonucleotide sequences useful for the discrimination between at least two genotypes of Cp. psittaci. A particular embodiment of this aspect of the invention relates to a method comprising the steps of a) providing a multiple alignment of a part of the genomic sequence of at least two Cp. psittaci genotypes, b) identifying regions which contain sequence differences within that part of the genomic sequence, c) synthesizing one or more oligonucleotides comprising a sequence wherein the above-identified sequence differences occur. Most particularly such a genomic sequence encodes a protein which causes pathogenicity, such as the OmpA protein. A particular embodiment of this aspect of the invention relates to a method whereby the part of the genomic sequence which is aligned to identify sequence differences comprises the sequence from about nucleotide 450 to about nucleotide 600 or from about nucleotide 900 to about nucleotide 1100 of the OmpA sequence corresponding to GB accession AF269281. Particular embodiments of this aspect of the invention relate to methods for generating oligonucleotide sequences useful for the discrimination between the genotype EB and another genotype of Cp. psittaci.

In yet a further aspect, the present invention provides oligonucleotides useful in the detection and/or identification of a Cp. psittaci genotype, most particularly the oligonucleotides selected from the group consisting of SEQ ID NO: 1′, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 24, SEQ ID NO: 25, and SEQ ID NO: 26. As detailed above, the present invention demonstrates how these oligonucleotides can be employed in methods which allow the detection and/or specific identification of a Cp. psittaci genotype

The methods and kits of the present invention, provide several advantages over the current detection and/or identification methods, such as easy sample collection methods, simple transport and storage requirements of the bacterial sample, rapid results, the possibility for automatisation, and a high sensitivity and specificity.

The present invention allows the genotype-specific detection of Cp. psittaci which is based on the identification of the presence of DNA of the bacterium. It allows the detection of the presence or absence of Cp. psittaci bacteria in a sample, independently of whether or not that sample comprises antibodies against bacteria of a previous infection. Thus, contrary to serotypic detection methods, the methods and kits of the present invention allow the detection and/or identification of an active infection.

Moreover, the methods and kits of the present invention allow the species- and genotype-specific detection of Cp. psittaci in a sample, e.g. a sample from a human, which contains at the same time one more bacterial infections caused by one or more organisms selected from the group consisting of Chlamydia trachomatis, Chlamydophila pneumonieae, and Chlamydophila abortus.

The present invention further makes it possible to determine or to confirm and follow-up the relationship between the occurrence of a certain genotype and the pathogenicity thereof.

### DETAILED DESCRIPTION

Definitions

“Genotype” as used in the present invention refers to the actual genetic composition of an organism as distinguished from its physical appearance (its phenotype). Thus while bacteria can have certain morphological properties which allow the determination of the organism up to the level of the genus, more subtle differences may occur which can only be attributed by sequence comparison of the whole genome or parts of the genome.

Bacteria belonging to the same genus, in this invention Cp. psittaci, can have differences in certain regions of the genome (in a preferred embodiment the OmpA gene) and will accordingly be classified in different genotypes.

The bacterium “Chlamydophila psittaci” (abbreviated as Cp. psittaci) belongs to the class of chlamidiae and is described in Skerman, V. B. D., McGowan, V., and Sneath, P. H. A. (editors). “Approved lists of bacterial names.” Int. J. Syst. Bacteriol. (1980) 30, 225-420. Synonyms which have been used are for this bacterium are Chlamydia psittaci, Chlamydozoon psittaci, Rickettsiaformis psittacosis, Ehrlichia psittaci and Rickettsia psittaci. In animals, Chlamydiaceae are capable of inducing a broad spectrum of symptoms like enteritis, urogenital infection, abortion, pneumonia, polyarthiritis, polyserositis, encephalitis and mastitis. Several genotypes are known, designated A to F. Of these genotypes, A, C and D have most often been associated with human psittacosis. However the occurrence of psittacosis is underestimated, as routine genotyping tools are not available.

“Sample” as used in the present application refers to either a solid or liquid substance. In the context of the present invention, the sample is preferably a body sample, i.e. a sample obtained from the animal or human body e.g. a part of the body, a body fluid or any excretion or waste product. According to the present invention the sample will contain sample DNA, i.e. DNA originating from the body from which the sample is obtained.

Samples include but without being limited thereto, blood, any cellular part of the body, skin, sputum, mouth, pharyngeal, conjunctival nose or vaginal swabs, urine, faecal samples, breath samples comprising aerosols of bacteria or bacteria particles, any type of tissue samples and biopts, such as lung, airsac, spleen or liver or other organs. Equally the methods of the present invention can be performed on bacterially infected cell cultures. The method can be performed on a sample of living bacteria but also on a sample comprising dead bacteria as long as DNA of the gene fragment to be amplified with the present method is available. Due to its sensitivity the sample can comprise less than 10.000, less than 1000, less than 100 or even about 10 or less than 10 Cp. psittaci bacteria or copies of the DNA to be amplified according to the method of the present invention.

“OmpA” in the present invention refers to the Outer Membrane Protein A of Cp. psittaci. As an illustration, Genbank accession AF269281 discloses the DNA and protein sequence of a strain of Chlamydophila psittaci. Partial sequences of Cp. psittaci OmpA from differing strains are presented in FIG. 1.

“Specific hybridisation” refers to the binding of a first nucleotide sequence with a DNA sequence which is completely or partially complementary thereto under stringent conditions. Nucleic acid hybridisation will be affected by such conditions as salt concentration, temperature, or organic solvents, in addition to the base composition, length of the complementary strands, and the number of nucleotide base mismatches between the hybridising nucleic acids, as will be readily appreciated by those skilled in the art. Stringent temperature conditions will generally include temperatures in excess of 30° C., typically in excess of 37° C., and preferably in excess of 45° C. Stringent salt conditions will ordinarily be less than 1000 mM, typically less than 500 mM, and preferably less than 200 mM. A oligonucleotide capable of hybridising specifically to the DNA of a particular genotype of Cp. psittaci thus refers to a genotype capable of hybridising thereto under stringent conditions.

“Essentially identical” used herein in the context of a sequence which is essentially identical to a specific sequence of a first (or competitor) oligonucleotide provided herein refers to a sequence which differs in one to three nucleotides from the specific sequence provided. The nucleotides differing are nucleotides selected by the skilled person in such a way that they do not affect the specificity of the oligonucleotide towards its genotype DNA. Most particularly the nucleotides selected are nucleotides which are identical within the DNA sequence of the different genotypes of Cp. psittaci. In the context of the sequences of PCR primers provided, ‘essentially identical’ refers to a sequence which differs from the provided sequence at maximally 5 nucleotides without affecting its ability to function as a PCR primer for the respective first oligonucleotide.

The present invention relates to the specific detection and/or identification of a Cp. psittaci genotype in a sample. According to particular embodiments of the present invention the sample originates from a human or from a non-human mammal, such as cattle, pigs, cats, dogs, birds (such as poultry exemplified by ducks, chicken, ostriches, turkeys, racing and urban pigeons, and pet birds (e.g. parrots)). The present invention provides a genotype-specific genetic assay for diagnosis and treatment-follow-up of Cp. psittaci infections from respiratory samples of animals, such as are particularly well-described in birds (ornithosis) and humans (psittacosis).

In one embodiment, the sample is a sample of a bird that is in a stage of development when the maternal immunity of the bird disappears and infection with Cp. psittaci is likely. In general, maternal antibody titers against an infection decline and are almost absent by 3 to 4 weeks of age. Turkeys normally experience two Cp. psittaci infection waves, one at 3 to 4 weeks and the second at 8 to 10 weeks of age. Accordingly the method of the present invention is advantageously performed on turkeys at these time points. Depending from animal species to species this time point on which the maternal immunity disappears varies. When the animal is a duck, the method is advantageously performed about 6 weeks after hatching of the egg. Further applications of the methods and kits of the present invention relate to the detection and/or identification of a Cp. psittaci genotype in a sample of an animal taken during or after the treatment against a Cp. psittaci infection or, in the case of e.g. poultry after the release from quarantine. The method of the present invention can be used in general to monitor the infection status of a poultry flock during production and for diminishing the risk of psittacosis in poultry workers. The method can also be used by public health officers to monitor the occurrence of the infection in risk groups as veterinarians and poultry workers. The method can be performed to evaluate the efficacy of treatment against a Cp. psittaci infection in both birds and humans. The method can also be used as a diagnostic control before releasing birds from quarantine or to monitor obligatory treatment during quarantine. The method can also be used to trace possible infection sources in case of human psittacosis outbreaks. The method can be used as taxonomic tool as it allows the detection of new genotypes. The method can also be used as a epidemiological tool for evaluating the relationship between the occurrence of a given genotype in birds and the risk of transmission to man as well as the relation between the occurrence of a genotype and the virulence thereof in both birds and mammals (especially humans).

Particular embodiments of the method of the invention are methods which comprise the steps of incubating a sample suspected of infection with Cp. psittaci with a first oligonucleotide, the first oligonucleotide being complementary to the DNA sequence of a genotype of Cp. psittaci allowing the hybridisation of a first oligonucleotide to DNA of Cp. psittaci present in a sample, and determining the binding of the first oligonucleotide within the sample. This last step ensures the identification of one or more genotypes of Cp. psittaci.

Particular embodiments of the methods of the present invention involve the use of different types of oligonucleotides. The ‘genotype-specific’ oligonucleotides also referred to as ‘first oligonucleotides’ used in the methods and kits of the present invention are oligonucleotides complementary to a DNA sequence of a Cp. psittaci genotype which is specific for this Cp. psittaci genotype and which is capable of hybridising specifically this specific sequence. In one embodiment of the invention capable of specifically hybridising refers to the ability of the oligonucleotide to hybridise specifically under hybridisation conditions which are commonly used during the elongation step of a PCR reaction.

The genotype-specific (first) oligonucleotides used in the context of the present invention can vary in length, between about 12 up to 30 or even 40 nucleotides, the proper length for an experiment being dependent on the technique used, the GC content of the probe used and the chance of non-specific binding of a probe to another target sequence. Specific embodiments of the invention, such as illustrated in the examples relate to probes of about 30 to 40 nucleotides. Differences can be envisaged wherein the probes are shorter or longer at their 3′ and/or 5′ end or are located more upstream or and/or downstream with respect to their target sequence (5, 10 15, 20 or more nucleotides). Particular embodiments of the first oligonucleotides suitable for use in the context of the present invention comprise or have the sequences in table 2 with SEQ ID NO: 1 (for genotype A), SEQ ID NO: 2 (for genotype B), SEQ ID NO: 3 (for genotype C), SEQ ID NO: 4 (for genotype D), SEQ ID NO: 5 (for genotype E), SEQ ID NO: 6 (for genotype F) and SEQ ID NO: 24 (for genotype EB). It will however be understood that sequences essentially identical to the sequences described herein can be designed for use in the context of the present invention.

In a particular embodiment the first oligonucleotide is labeled with a chromophoric group at its 5′ and with a quencher group at its 3′ end, in order to be suitable for use in a quantitative PCR method (e.g. so called “taqman”). Suitable labels include but are not limited to e.g. the fluorescent indicator molecules selected from the group consisting of fluorescein, rhodamine, texas red, FAM, JOE, TAMRA, ROX, HEX, TET, Cy3, Cy3.5, Cy5, Cy5.5, IRD40, IRD41 and BODIPY.

The binding of an oligonucleotide to DNA present in a sample can be determined via a variety of techniques such as southern or northern hybridisation and chromatography under denaturing conditions. In one embodiment of the invention, the binding of an oligonucleotide can be determined by evaluating the binding of an identical non-labeled oligonucleotide for the same binding site. e.g. replacement of a chromogenic probe by a non chromogenic probe or vice versa. In a particular embodiment, the replacement of the non-chromogenic probe occurs during a PCR reaction wherein a quencher group is removed from a probe by DNA polymerase.

According to a specific embodiment, the methods of the invention are quantitative real-time PCR assays. It is demonstrated herein that the assays of the invention meet the criteria proposed for a validated assay, as both new real-time PCR assays were compared with other assays such as ompA sequencing, ompA RFLP and MOMP serotyping. Real-time PCR technology offers a new diagnostic approach which allows amplicon quantification in one step via specific hybridisation, without the need to open tubes, minimising the risk of cross-contamination for further experiments in this way.

The present invention further presents a method of generating genotype specific antibodies, which are derived from peptides having a sequence located within one of the sequences depicted in FIG. 1. Oligopeptides having a unique sequence for a certain genotype are used for the generation of antibodies.

According to a specific aspect of the methods and kits of the present invention second or competitor probes are used in combination with the first genotype-specific oligonucleotides of the invention. The second or competitor probes of the present invention are genotype-specific probes directed against another Cp. psittaci genotype DNA which genotype is different from the one which is envisaged to be detected and prevents non-specific binding of the first oligonucleotide according to the present invention to said other Cp. psittaci genotype. Thus, according to this aspect, the method comprises

incubating the sample in addition to the first oligonucleotide with a second oligonucleotide (so called competitor) and determining the binding of the first oligonucleotide to DNA within the sample. Depending from the first oligonucleotide used, different competitors can be suitable for ensuring the increased specificity of the detection. According to one embodiment the first oligonucleotide corresponds to one of the sequences selected from SEQ ID NO: 1 to 6 or 24 described herein and the competitor oligonucleotide corresponds to a sequence comprising the nucleotide sequence in the OmpA gene which can be aligned with another one of the sequences of SEQ ID NO: 1 to 6 or 24. Most particularly, for the detection of genotype A, the first oligonucleotide is corresponds to SEQ ID NO: 1 and the competitor oligonucleotide is a sequence corresponding to SEQ ID NO: 1 within the sequence of the OmpA gene of the genotype B, C, D, E, F or EB (after alignment of the OmpA sequence of genotype A to that of genotype B, C, D, E, F or EB). The following embodiments represent examples of suitable combinations of competitors and probes:
the second oligonucleotide comprises the sequence of SEQ ID NO: 8, and the first oligonucleotide comprises the sequence of SEQ ID NO: 1;
the second oligonucleotide comprises the sequence of SEQ ID NO: 7, and the first oligonucleotide comprises the sequence of SEQ ID NO: 2;
the second oligonucleotide comprises the sequence of SEQ ID NO: 10, and wherein the first oligonucleotide comprises the sequence of SEQ ID NO: 2;
the second oligonucleotide comprises the sequence of SEQ ID NO: 9, and the first oligonucleotide comprises the sequence of SEQ ID NO: 5;
the second oligonucleotide comprises the sequence of SEQ ID NO: 11, and the first oligonucleotide comprises the sequence of SEQ IUD NO: 5.

Again, it will however be understood that sequences essentially identical to the sequences described herein can be designed for use in the context of the present invention. Moreover, it will be understood that further competitor oligonucleotides can be designed by the skilled person to avoid non-specific hybridisation of a first oligonucleotide of the invention with a DNA sequence of a genotype other than the one against which it is directed.

As detailed above, according to a particular embodiment, the method of detection of the binding is PCR. In a preferred embodiment the binding of a first and/or binding of a second oligonucleotide is determined by PCR amplification with a forward and a reverse primer, wherein the forward and reverse primer are located about 1, 5, 10, 20, 50 to 100 bp 3′ and 5′ from the first or second oligonucleotide. The PCR may be real-time PCR. Multiplexing can be used to reduce time.

The following embodiments represent examples of suitable pairs of forward and reverse primers for respective first oligonucleotides:
primers comprising or containing the sequence of SEQ ID NO: 12 and SEQ ID NO: 13 when the first oligonucleotide comprises or contains the sequence of SEQ ID NO: 1.
primers comprising or containing the sequence of SEQ ID NO: 14 and SEQ ID NO: 15 when the first oligonucleotide comprises or contains the sequence of SEQ ID NO: 2.
primers comprising or containing the sequence of SEQ ID NO: 16 and SEQ ID NO: 17 when the first oligonucleotide comprises or contains the sequence of SEQ ID NO: 3.
primers comprising or containing the sequence of SEQ ID NO: 18 and SEQ ID NO: 19 when the first oligonucleotide comprises or contains the sequence of SEQ ID NO: 4.
primers comprising or containing the sequence of SEQ ID NO: 20 and SEQ ID NO: 21 when the first oligonucleotide comprises or contains the sequence of SEQ ID NO: 5.
primers comprising or containing the sequence of SEQ ID NO: 22 and SEQ ID NO: 23 when the first oligonucleotide comprises or contains the sequence of SEQ ID NO: 6.
primers comprising or containing the sequence of: SEQ ID NO: 25 and SEQ ID NO: 26 when the first oligonucleotide comprises or contains the sequence of SEQ ID NO: 24.

In another aspect the invention relates to isolated oligonucleotides comprising a or containing a sequence selected from the group of consisting of SEQ ID NO: 1, SEQ ID NO: 2, SEQ ID NO: 3, SEQ ID NO: 4, SEQ ID NO: 5, SEQ ID NO: 6, SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23 SEQ ID NO: 24, SEQ ID NO: 25 and SEQ ID NO: 26, and sequences which are essentially identical thereto.

In another aspect the invention relates to a diagnostic kit comprising one or more oligonucleotides capable of specifically hybridizing to a DNA sequence of a genotype of Cp. psittaci. According to a particular embodiment the diagnostic kits comprise one or more of the first genotype-specific oligonucleotides capable of hybridising specifically to a genotype of the Cp. psittaci, most specifically one of the oligonucleotides selected from the group consisting of SEQ ID NO: 1 to 6 and SEQ ID NO: 24. According to a further embodiment the kit can additionally comprise one or more competitor probes, more specifically, one or more of the competitor oligonucleotides selected from the group consisting of SEQ ID NO: 8 to 11. Additionally or alternatively the kits of the present invention can comprise two primers, more particularly primer pairs selected from the group consisting of SEQ ID NO: 12 and 13, SEQ ID NO: 14 and 15, SEQ ID NO: 16 and 17, SEQ ID NO: 18 and 19, SEQ ID NO: 20 and 21, SEQ ID NO: 22 and 23, SEQ ID NO: 25 and 26. The kit can be further supplemented with e.g. reference strains of Cp. psittaci bacteria, plasmids containing a complete or partial OmpA DNA sequence of reference genotypes, or antibodies against Cp. psittaci. Other components can be bacteria or DNA samples of bacteria closely related to Cp. psittaci.

Another aspect of the invention relates to a novel strain of a Cp. psittaci bacterium, designated as Cp. psittaci genotype EB. This novel strain is characterized in that its genome comprises the specific sequence of the OmpA gene depicted in SEQ ID NO: 51. As detailed herein, the identification of this novel strain allows a more specific identification of the genotypic strains of Cp. psittaci in a sample. The present invention further provides EB-genotype-specific sequence SEQ ID NO: 51 and the use of this sequence and (EB-specific) fragments thereof in different applications, such as, but not limited to the specific detection of Cp. psittaci EB genotype, the generation of antibodies against corresponding amino acids sequences, etc.

Another aspect of the invention relates to a method of generating oligonucleotide sequences useful for the discrimination between at least two genotypes, in the detection of Cp. psittaci, the method comprising the steps of: a) providing a (multiple) alignment of a part of the genomic sequence of the at least two Cp. psittaci genotypes, b) identifying regions which contain sequence differences within said part of the genomic sequence, and c) synthesizing one or more oligonucleotides comprising a sequence wherein said sequences differences occur. According to one embodiment the genomic sequence used comprises a sequence for a gene encoding a protein which causes pathogenicity. In another embodiment, one of the at least two genotypes of Cp. psittaci is of the genotype EB.

The present invention is further illustrated with the following Figures and Examples, not intended to limit the scope of the invention.

### FIGURE LEGENDS

FIG. 1: alignment of parts of the OmpA sequence of different Cp. psittaci strains (genotypes) with probes (double underlined) and forward and reverse primers (underlined) in accordance with an embodiment of the present invention.

FIG. 2: genotype-specific standard curves obtained with the GeneAmp 5700 apparatus.

FIG. 3: quantitative PCR results of VS-study mixed infections

### EXAMPLE 1

### General Methodology

Isolates and cell cultures. Cp. psittaci genotype A to F plus E/B reference strains 90/1051, 41A12, GD, 7344/2, 3759/2, 7778B15 and WS/RT/E30 (Table 1), were grown in Buffalo Green Monkey (BGM) cells. Infected monolayers were disrupted by freezing and thawing followed by ultrasonic treatment for 1 minute in a tabletop sonicator (Bransonic 12, BIOMEDevice, San Pablo, Calif., USA). The cell culture harvest was centrifuged for 10 min (1,000×g, 4° C.) to remove cellular fragments and subsequently concentrated by ultracentrifugation for 1 hour (45,000×g, 4° C.). Bacterial pellets were resuspended in Sucrose Phosphate Glutamate buffer (SPG) (218 mM sucrose, 38 mM KH₂PO₄, 7 mM K₂HPO₄, 5 mM L-glutamic acid) at a volume of 1 to 100 of the original culture volume and stored at −80° C. until use.

DNA extraction. Genomic DNA was prepared as follows. 200 μl cell culture harvest was centrifuged for 30 min at RT (16,000×g). The supernatant was discarded and the pellet resuspended in 199 μl SET buffer pH 7,5 (0.05 M Tris, 0.01 M EDTA, 1% SDS) supplemented with 1 μl Proteinase K (20 mg/ml, Promega, Madison, Wis., USA). Samples were incubated at 37° C. for 30 min and subsequently boiled for 10 min to inactivate the enzyme.

Genotype-specific reference plasmid constructions. The ompA gene of the genotype A to F plus E/B reference strains (Table 1) was amplified resulting in a fragment of 1,065 to 1,098 bp depending on the genotype. Primers were chosen from the highly conserved regions of the published ompA sequences of C. trachomatis and Cp. psittaci. Amplification of the ompA gene was accomplished using the genoI [SEQ ID NO:52] and genoII [SEQ ID NO: 53] primers (Table 2) syntesized by Invitrogen. Thirty-five cycles of 1 min denaturation at 95° C., 2 min annealing at 55° C. and 3 min extension at 72° C. were completed in a Perkin Elmer GeneAmp 9600 after an initial denaturation of 5 min at 95° C. and followed by 5 min end annealing at 72° C.

TABLE 1
Cp. psittaci reference plasmids

| | | | | Geno- |
|---|---|---|---|---|
| Plasmid | Strain | Country (year) | Host | type |
| 22A | 90/1051 | Belgium (1990) | Amazona sp. | A |
| 29B | 41A12 | Belgium (2001) | Meleagris gallopavo | B |
| 45A | GD | Gemany (1960) | Anas platyrhyncos | C |
| 19A | 7344/2 | Italy (1997) | Columba livia ^{a} | D |
| 17A | 3759/2 | Italy (1999) | Columba livia ^{a} | E |
| 32B | 7778B15 | Belgium (2001) | Meleagris gallopavo | F |
| 35A | WS/RT/E30 | Germany (2001) | Anas platyrhyncos | EB |

^{a} Isolated from an urban pigeon

TABLE 2

PCR primers, probes and competitors for geno-
type specific detection of Cp psittaci.

| | | | |
|---|---|---|---|
| | | Melt- | |
| | | ing | SEQ |
| | | Point | ID. |
| Oligo | Sequence (5′-3′) | (° C.) | NO: |

| | | | |
|---|---|---|---|
| Genotype A | Fam-CTACCGATCTTCCAACGCAACTTC- | 69 | 1 |
| probe | CTAACG-Tamra | | |
| | (or other chromphoric and/or | | |
| | quencher group) | | |
| Genotype A | 5′-GGTTTTCAGCTGCAAGCTCAA-3′ | 59 | 12 |
| forward | | | |
| Genotype A | 5′-CCACAACACCTTGGGTAATGC-3′ | 59 | 13 |
| Reverse | | | |
| Genotype B | Fam- | 69 | 2 |
| probe | TCTACCGATCTTCCAATGCAACTTC- | | |
| | CTAACGTATamra | | |
| | (or other chromphoric and/or | | |
| | quencher group) | | |
| Genotype B | 5′- | 59 | 14 |
| forward | AATAGGGTTTTCAGCTACCAACTCAA-3′ | | |
| Genotype B | 5′-CCACAACACCTTGGGTAATGC-3′ | 59 | 15 |
| reverse | | | |
| Genotype C | Fam-TCTGCTGTTATGAACTTGACCAC- | 69 | 3 |
| probe | ATGGAACC-Tamra | | |
| | (or other chromphoric and/or | | |
| | quencher group) | | |
| Genotype C | 5′-GCATCGCTCAACCTAAATTGG-3′ | 58 | 16 |
| forward | | | |
| Genotype C | 5′-ATTGTGGCTTCCCCTAAAAGG-3′ | 58 | 17 |
| reverse | | | |
| Genotype D | Fam-AGGAAAGGCCACAACTGTCGACGG- | 68 | 4 |
| probe | Tamra | | |
| | (or other chromphoric and/or | | |
| | quencher group) | | |
| Genotype D | 5′-AACCACTTGGAACCCAACACTTT-3′ | 60 | 18 |
| forward | | | |
| Genotype D | 5′-CGAAGCAAGTTGTAAGAAGTCAG- | 60 | 19 |
| reverse | AGTAA-3′ | | |
| Genotype E | Fam- | 68 | 5 |
| probe | TACTTTGCCCAATAATGGTGGTAAG- | | |
| | GATGTTCTATC-Tamra | | |
| Genotype E | 5′-CCAAGCCTTCTAGGATCAAGGA-3′ | 59 | 20 |
| forward | | | |
| Genotype E | 5′-CGAAGCAATTTGCAAGACATCA-3′ | 60 | 21 |
| reverse | | | |
| Genotype F | Fam-CATCGCTCAACCTAAATTAGCCGC- | 68 | 6 |
| probe | TGC-Tamra | | |
| Genotype F | 5′- | 59 | 22 |
| forward | GCAACTTTTGATGCTGACTCTATCC-3′ | | |
| Genotype F | 5′- | 58 | 23 |
| Reverse | GTTCCATGTGGTCAAGTTCAAAAC-3′ | | |
| Genotype EB | 5′-CCAAGCCTTCTAGGATCAACCA-3′ | | 24 |
| Probe | | | |
| Genotype EB | 5′-TGCTTTGCCCAATAATGCTG-3′ | | 25 |
| Forward | | | |
| Genotype EB | 5′- | | 26 |
| Reverse | AAGGATGTTCTATCTGATGTCTTGCA-3′ | | |
| Genotype A | 5′-CTACCGATCTTCCAATGCAACTT- | | 8 |
| competitor | CCTAACG-3′ | | |
| B | | | |
| CpPsGAcomB | | | |
| Genotype B | 5′-TCTACCGATCCTTCCAACGCAAC- | | 7 |
| competitor | TTCCTAACGTA-3′ | | |
| A | | | |
| CpPsGBcomA | | | |
| Genotype B | 5′-TCTACCGAGCTTCCAATGCAA- | | 10 |
| competitor | CTTCCTAACGTA-3′ | | |
| E + E/B | | | |
| CpPsGBcom | | | |
| E + E/B | | | |
| genotype E | 5′- | 69 | 9 |
| competitor | TGCTTTGCCCAATAATGCTGGTAAGG- | | |
| E/B | ATGTTCTATC 3′ | | |
| CpPsGEcomEB | | | |
| Genotype E | 5′-TGCTTTGCCCAATAATAGTGGTA- | | 11 |
| competitor | AGGATGTTCTATC 3′ | | |
| AB | | | |
| CpPsGEcomAB | | | |
| GenoI | 5′-ATGAAAAAACTCTTGAAATCG-3′ | 55 | 52 |
| GenoII | 5′-ACAAGCTTTTCTAGACTTCAT-3′ | 55 | 53 |

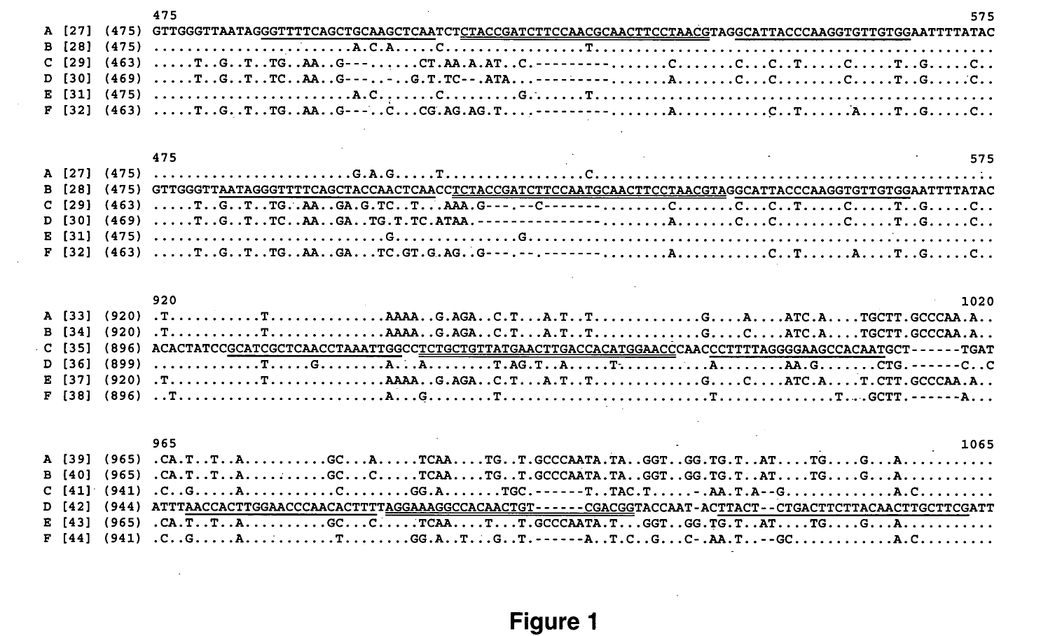

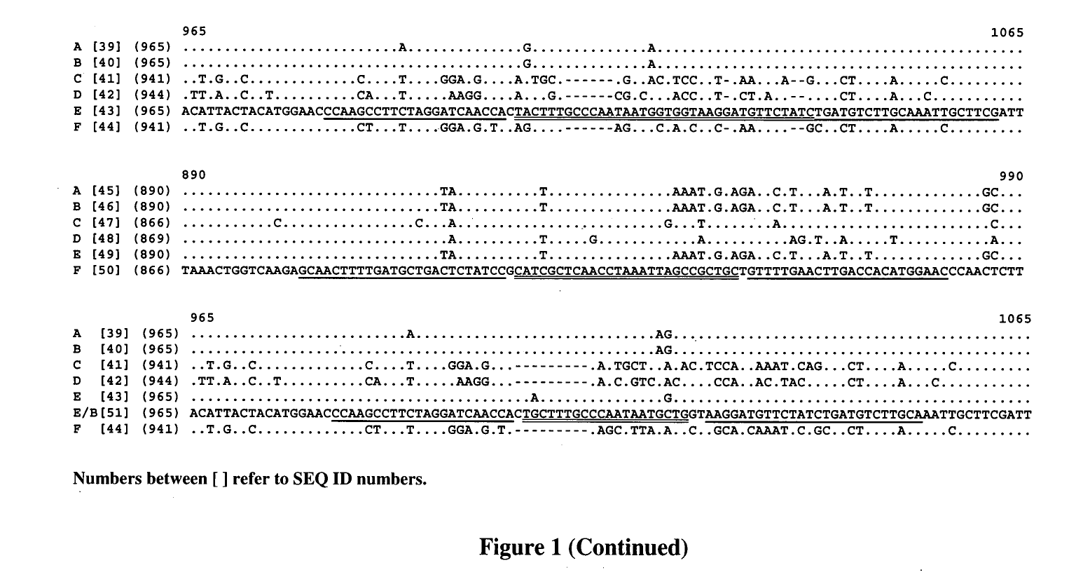

Quantitative ompA Genotype specific real-time PCR. Cp. psittaci genotype specific PCR primers were selected from the variable segments of the ompA gene with primer express software (Applied biosystems) and synthesized by Invitrogen. The PCR products generated were between 78 and 85 bp depending on the genotype. Sequences of the primers and TaqMan probes (synthesized by Applied Biosystems) for the different genotypes are presented in Table 2. The genotype specific probes were 5′ labelled with 6-carboxyfluorescein (FAM) as the reporter dye and with 6-carboxythetramethylrhodamine (TAMRA) at the 3′ end as the quencher. Other dye-quencher combinations can be used as alternatives. A sequence alignment of parts of the OmpA gene and the probes being used are shown in FIG. 1. For the A, B and E genotypes, competitor oligo's were used to enhance the specificity of the probe. Forward and reverse primers and probes were tested in concentrations of 50, 150, 300 and 900 nM, with and without adding the competitor DNA (50 nM or 150 nM), supplemented with purified genomic DNA of the six genotype reference strains. Best results were achieved with forward and reverse primer concentrations of 300 nM, a probe concentration of 300 nM and where applicable, a competitor concentration of 50 nM. Cycling conditions were those suggested by the manufacturer and all default program settings were used. PCR was performed in ABI PRISM® optical tubes (Applied Biosystems), with the reaction mixtures consisting of 25 μl of the TaqMan universal Master mix including dUTP and uracyl N-glycosylase (AmpErase UNG; Applied Biosystems), in a total reaction volume of 50 μl. Amplification and detection of the PCR product was performed with an ABI GeneAmp 5700 sequence detection instrument (Applied Biosystems), using all default program settings. Cycling conditions were as follows: after 2 min 50° C. and 10 min at 95° C., the samples were submitted for 40 cycles, each consisting of an initial denaturation step at 95° C. for 15 s followed by a step at 60° C. for annealing and extension for one minute. The PCR products were detected as an increase in fluorescence during the PCR extension phase when the probe was cleaved by the 5′ exonuclease activity of the Taq DNA polymerase. Standard graphs of the Ct values obtained from serial dilutions of purified reference plasmids (10⁸ to 10¹) were constructed. Ct values for unknown clinical samples were plotted against the standard graphs for plasmids. Finally, the amount of the different Cp. psittaci genotypes present in the clinical samples (N₀) was. In addition, DNA from each clinical sample was tested in the presence of Cp. psittaci DNA (50 ompA copies for each genotype) to check for PCR inhibitors by comparing the amplification plots for the samples with and without this internal controls.

Identical or similar settings can be used in apparatus from other manufactures in order to reproduce the disclosure of the present invention.

Positive controls and constructed test samples. Mixtures of plasmids with OmpA of known concentration can be used as a model for mixed cultures of Cp. psittaci because OmpA occurs as single copy gene in the bacterium.

Clinical samples. Ornithosis/psittacosis study. In an experiment with five groups of SPF turkeys (5.07, 5.09, 5.10, 5.11 and 5.12), animals of each group were dying due to an unknown cause, having severe respiratory symptoms. Pharyngeal swabs from each group of animals were collected by serial passage through the five animals in each group, as well as from the veterinarian who took care of them to verify whether Cp. psittaci was the causative agent. A second swab of the veterinarian was taken two weeks later. Swabs were shaken in 1 ml sucrose phosphate glutamate buffer (SPG, 218 mM sucrose, 38 mM KH₂PO₄, 7 mM K₂HPO₄, 5 mM L-glutamic acid). One-day-old HeLa monolayers were inoculated with the supernatant and examined with the Chlamydia Imagen kit (DakoCytomation) according to the manufacturers instructions.

In parallel, 100 μl suspension was centrifuged (10 min2700×g) and used for DNA extractions with the SET-method.

Longitudinal study. A longitudinal study was performed on three turkey farms in order to examine the kinetics of avian pneumovirus (APV), Ornithobacterium rhinotracheale (ORT), M. gallisepticum, M. meleagridis and Cp. psittaci infections from day one until slaughter. Pharyngeal swabs from week 3, 6, 8, 12 and 15 after hatching were used for DNA extraction with the SET-method to quantify the presence of Cp. psittaci and to compare this result with the antibody response of the animals during the infection as determined by ELISA VS-study. In a previous study performing whole ompA sequencing of several clones per isolate revealed the presence of 5 mixed-genotype infections on a total of 21 isolates.

Genomic DNA extractions of these isolates were used to verify the presence of the genotypes found by sequencing with the genotype specific RT-PCR-reactions.

### EXAMPLE 2

### Genotype Specific Identification of Cp. psittaci

The present invention demonstrates for the fist time the use of real time PCR technology to detect seven different avian Cp. psittaci genotypes in human and animal samples and offers the possibility to discover new Cp. psittaci genotypes.

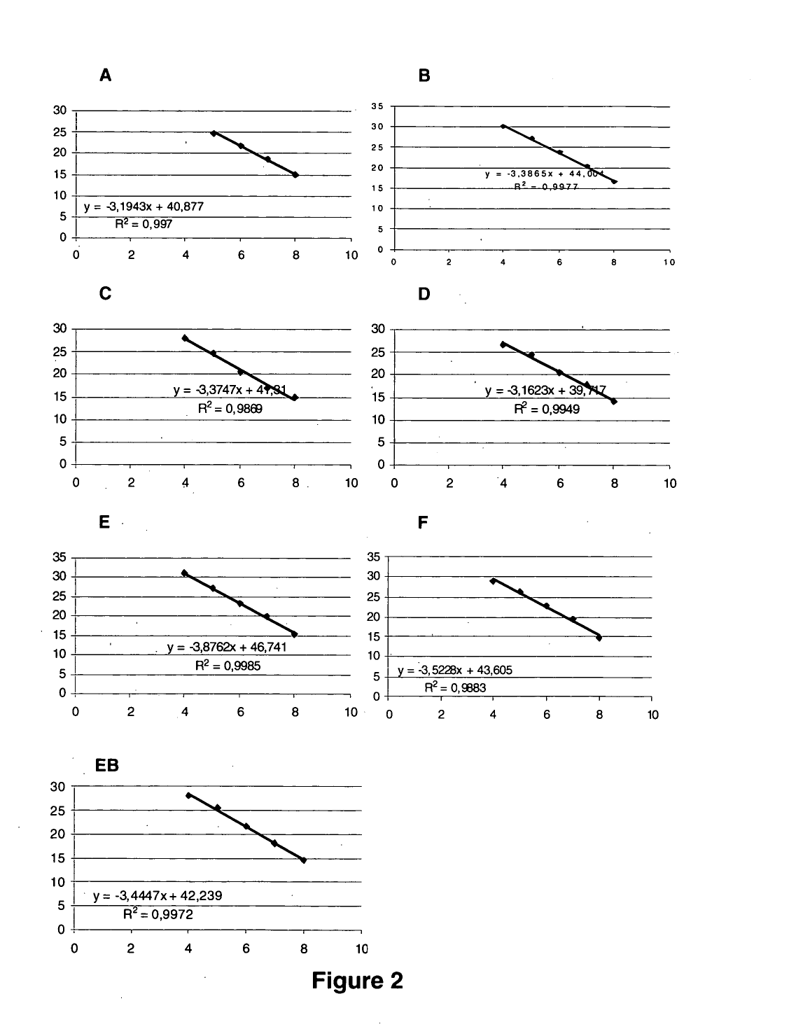

Using genotype specific reference plasmids, all seven PCR's (A to EB) are able to detect 10 copies of plasmid per μl. Standard curves could be made from 10⁸ to 10⁵ copies per μl with almost ideal slopes around −3,3 and correlation coefficients higher then 98,5% (FIG. 2). The highest dilutions were not taken into account for the regression because the reproducibility was too low, they reached the threshold around the same cycle or only after cycle 40.

The competitors which have been used in the PCR methods of the present invention are oligonucleotides without a fluorescent signal that go in competition with probes that bind to the target sequence. In Fluorescence In Situ Hybridization (FISH) they are frequently used to enhance specific binding of the probes by blocking the possible probe sites on contaminating DNA. Competitors were until now never used in RT-PCR. This principle disclosed in this invention is applicable in any type of PCR reaction, wherein a probe is used which resides between the forward and reverse primer and wherein a further oligonucleotide is being used which competes with the probe for binding to the template DNA.

When investigating the primer and probe specificity of the reactions by preparing a mixture of 1/10 dilutions of genomic DNA extracts of the different genotypes plus undiluted, 1/100 and 1/1000 diluted material of the specific genotype, the results indicated that the C, D and F primers and probes did not render any significant reaction with the 1/10 dilution of the other genotype extracts, but the A, B, E and EB probes on the other hand did react with the other genomic material present. The development of genotype specific competitors allowed to differentiate all seven genotypes when added in a concentration of 50 nM. Competitor sequences are shown in Table 2.

### EXAMPLE 3

### Genotype Determination

Genotype A. The Chlamydophila psittaci genotype A specific competitor for binding on genotype B (CpPsGAScomB) [SEQ ID NO:8] has to be added to the reaction mixture to prevent false positive results if genotype B is possibly present in the sample. When added, the competitor will bind the genotype B DNA, leaving the probe only the binding site on genotype A, if present. As the competitor sequence is complementary to the genotype B sequence, the affinity is higher for this genotype, while the probe off course preferentially binds genotype A.

Genotype B. In genotype B determination, an elevated temperature can enhance the probe specificity: a specific reactions with genotypes E and EB disappear when the reaction is carried out at 63° C. in stead of 60° C. Addition of the competitor for genotype A material CpPsGBScomA [SEQ ID NO:7] will prevent false positive reactions if genotype A material is present.

Genotype E. Addition of both CpPsGEScomA/B [SEQ ID NO:11] (competitor to prevent binding of probe E to genotype A and to genotype B) and CpPsGEScomEB [SEQ ID NO:9] (to prevent binding to EB) in equal concentrations of 50 nM prevent reaction with A and B efficiently, while the false positive signal EB comes several cycli later and the intensity is of it is reduced to 25% of the specific E signal.

### EXAMPLE 4

### Genotype Specific Detection of Cp. psittaci on Clinical Samples

Ornithosis/Psittacosis Study.

Samples 5.07, 5.09, 5.10, 5.11 and 5.12 from the turkeys as well as the two samples of the veterinarian (V1 and V2) were all positive in DIF three days post inoculation. When the genotype specific RT reactions were carried out directly on the sample resuspended in SPG, there was no reaction. Addition of the internal controls (50 copies/μl of the reference plasmids) proved that this was due to inhibition of the reaction. After SET-DNA extraction, reactions were done again and results showed that all turkeys were infected with the genotypes D, F and EB. On the same moment, the veterinarian already seemed infected with genotypes D and EB. The second sample of the veterinarian showed the genotypes D, F and EB to be present. Standard curves were made with 10⁷, 10⁵ and 10³ reference plasmids per μl on an ABI prism 7000 and Ct's of the samples were determined and plotted against the standard curves to determine the number of particles for each genotype. Results are shown in Table 3. These results show the zoonotic effect of Cp. psittaci: although there were no visible clinical symptoms, the veterinarian became infected with the same genotype strains as the turkeys. On the first timepoint genotype F was not yet detected, but sample V2 shows that genotype F had the chance to multiplicate in an incubation time of two weeks (Table 3).

TABLE 3
Quantification analysis ornithosis/psittacosis study on samples
of birds (5.07, 5.09, 5.10, 5.11, 5.12) and humans (V1/V2)

| geno- | 3 point | 5.07 | | | 5.09 | | | 5.10 | | |
|---|---|---|---|---|---|---|---|---|---|---|
| type | std curve | C_{T} | X-value | Copies/μl | C_{T} | X-value | copies/μl | C_{T} | X-value | copies/μl |
| D | Y = −2.74X + 37.62 | 34.37 | 1.18 | 15 | 31.43 | 2.26 | 182 | 33.1 | 1.65 | 45 |
| F | Y = −3.20X + 40.89 | 35.18 | 1.78 | 61 | 29.55 | 3.54 | 3497 | 32.22 | 2.70 | 512 |
| EB | Y = −2.7X + 37.37 | 31.3 | 2.25 | 177 | 29.57 | 2.89 | 772 | 31.28 | 2.25 | 180 |

| Geno- | 3 point | 5.11 | | | 5.12 | | | V1/V2 | | |
|---|---|---|---|---|---|---|---|---|---|---|
| type | std curve | C_{T} | X-value | copies/μl | C_{T} | X-value | copies/μl | C_{T} | X-value | copies/μl |
| D | Y = −2.74X + 37.62 | 32.97 | 1.70 | 50 | 34.69 | 1.07 | 12 | 34.49/ | 1.14/ | 14/ |
| | | | | | | | | 35.99 | 0.59 | 6 |
| F | Y = −3.20X + 40.89 | 35.27 | 1.76 | 57 | 35.88 | 1.57 | 37 | —/ | —/ | —/ |
| | | | | | | | | 33.4 | 2.34 | 220 |
| EB | Y = −2.7X + 37.37 | 31.85 | 2.04 | 110 | 31.34 | 2.23 | 171 | 31.99/ | 1.99/ | 98/ |
| | | | | | | | | 31.22 | 2.28 | 189 |

Longitudinal study. DNA extracts from swabs after 3, 6, 8, 12 and 15 weeks after hatching were screened in the species specific PCR in a Perkin Elmer GeneAmp 9600 apparatus (Wellesley, Mass., USA) without SybrGreen. All samples showed the characteristic 151 bp amplicon, already proving that the animals were infected with a Cp. psittaci genotype B strain and that two infections (week 6 and 12) were found on the farm. A genotype B standard curve with 10⁷, 10⁵ and 10³ reference plasmids per μl was made on an ABI prism 7000 and Ct's of the samples were determined and plotted against the standard curves to determine the number of particles. The genotype B specific real time PCR could prove that the high antibody responses were indeed correlated with a tenfold increase in Cp. psittaci genotype B (see week 6 and 12, in Table 4).

TABLE 4
Quantification analysis of the longitudinal study

| | Week | | | | | | | |
|---|---|---|---|---|---|---|---|---|
| | 0 | 1 | 2 | 3 | 4 | 5 | 6 | 7 |
| Titer | 3072 | 768 | 768 | 1536 | 768 | / | 3072 | / |
| Copies/μl | /^{a} | / | 24 | / | / | / | 217 | / |

| | Week | | | | | | | |
|---|---|---|---|---|---|---|---|---|
| | 8 | 9 | 10 | 11 | 12 | 13 | 14 | 15 |
| Titer | 1536 | / | 768 | / | 3072 | / | 768 | / |
| Copies/μl | 33 | / | / | / | 238 | / | / | 19 |

^{a}/ not available; calculation was done with the genotype B standard curve y = −2.92x + 39.53 with y = Ct and x = log (copies/μl)

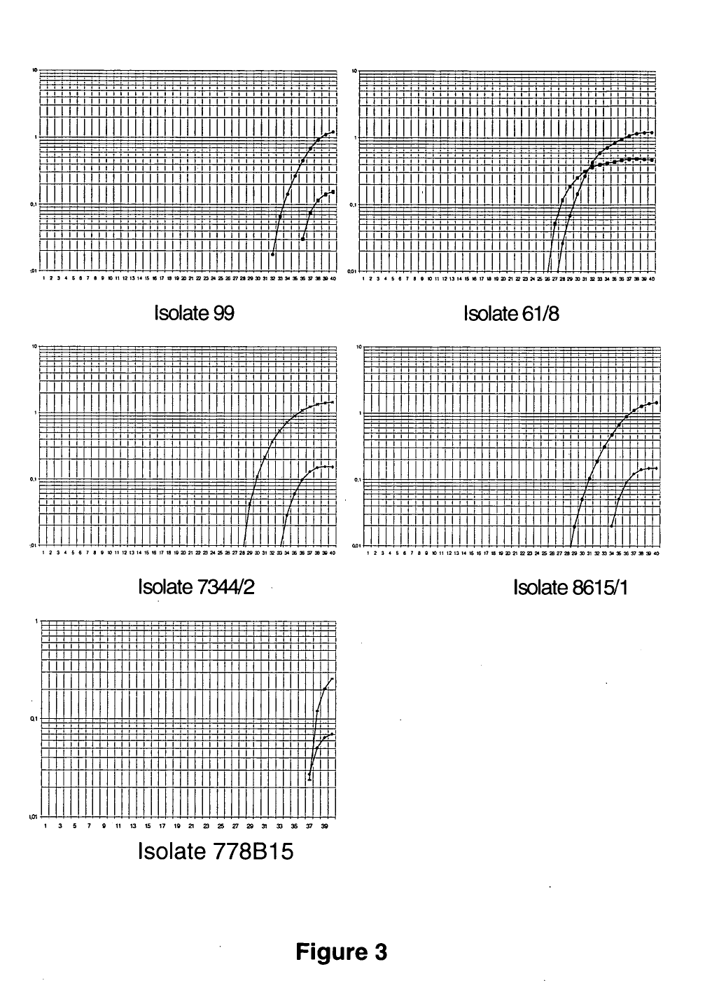

VS-study. Isolates revealing mixed infections were submitted to the genotype specific real-time pcr reactions to confirm the presence of the different genotypes indicated by the whole ompA sequencing. Table 5 shows that all mixed infections could be detected easily and moreover, quantified using the Ct values determined on the graphs presented in FIG. 3 and the standard curves of FIG. 2. The genotype that is less abundant remains undetected in four of the five cases.

In addition to the specificity of the quantitative PCR method to discriminate genotypes, the specificity was also tested on DNA extracted from other bacterial species commonly found in the avian and human respiratory tract as well as on DNA extracted from avian (HD11) and (Hela) cells. No amplified DNA prodcuts were detected.

TABLE 5
Quantification analysis VS-study

| | | | MOMP | | | |
|---|---|---|---|---|---|---|
| | OmpA | OmpA | sero- | | | |
| Isolate | sequencing | RFLP | typing | Ct | X | N₀ ^{a} |
| 99 | A (01B) + | A + E | B | 35.75 | 1.605046 | 40 |
| | E/B (01A + | | | 32.05 | 2.957877 | 907 |
| | 01D) | | | | | |
| 61/8 | A (11D) + | A + E | A + B | 26.26 | 4.575963 | 37667 |
| | E/B (11C) | | | 27.68 | 4.226493 | 16846 |
| 7344/2 | B (19D) + | B + D | B | 33.59 | 3.075151 | 1189 |
| | D (19B) | | | 28.37 | 3.588211 | 3874 |
| 8615/1 | B (20A + | B + E | B | 34 | 2.954082 | 900 |
| | 20C) + | | | | | |
| | E/B (20D) | | | 29.01 | 3.840392 | 6925 |
| 7778B15 | B (32A) + | B + F | B | 36.74 | 2.144987 | 140 |
| | F (32D + | | | 36.96 | 1.886284 | 77 |
| | 32F) | | | | | |

^{a} N₀were calculated using the regression curves presented in FIG. 2

What is claimed is:

1. An ex vivo or in vitro method for the identification of the presence of DNA of a genotype of Cp. psittaci in a sample, said method comprising the steps of:
incubating said sample with a first oligonucleotide which is capable of specifically hybridising to DNA of a genotype of Cp. psittaci, and,
determining the binding of said first oligonucleotide to DNA within said sample, which binding is indicative of the presence of DNA of a genotype of Cp. psittaci in said sample.

2. The method according to claim 1, wherein said first nucleotide comprises a sequence of at least 15 nucleotides of the OmpA gene of one of the Cp. psittaci genotypes.

3. The method according to claim 1, wherein said first nucleotide comprises a sequence of at least 15 nucleotides within the region from about nucleotide 450 to about nucleotide 600 or from about nucleotide 900 to about 1100 of the OmpA sequence corresponding to GB accession AF269281.

4. The method of claim 1, wherein said genotype of Cp. psittaci is selected from genotypes A, B, C, D, E, F and EB.

5. The method according to claim 1, wherein said first oligonucleotide is labeled with a chromophoric group at its 5′ and with a quencher group at its 3′ end.

6. The method of claim 1, wherein said sample is incubated with more than one first oligonucleotide, and wherein each of said more than one first nucleotide is capable of hybridising to DNA of a genotype of Cp. psittaci.

7. The method according to claim 1, wherein the first oligonucleotide comprises a sequence selected from the group consisting of:
sequence corresponding to SEQ ID NO: 1 for genotype A,
sequence corresponding SEQ ID NO: 2, for genotype B,
sequence corresponding SEQ ID NO: 3 for genotype C,
sequence corresponding SEQ ID NO: 4, for genotype D,
sequence corresponding SEQ ID NO: 5, for genotype E,
sequence corresponding SEQ ID NO: 6, for genotype F and
sequence corresponding SEQ ID NO: 24, for genotype EB,
or a sequence essentially identical thereto capable of hybridizing specifically to said genotype.

8. The method according to claim 1, further characterized in that said sample is additionally incubated with at least one second oligonucleotide, said second oligonucleotide being a competitor for the hybridisation of said first oligonucleotide to DNA of another genotype of Cp. psittaci.

9. The method according to claim 8, wherein said competitor probe comprises a sequence corresponding to a sequence within the DNA of a genotype of Cp. psittaci other than the genotype to which the first probe is directed, which can be aligned with the sequence of said first probe.

10. The method according to claim 8, wherein said first and said second oligonucleotide are selected from the group consisting of:
said second oligonucleotide comprises the sequence of SEQ ID NO: 8, and said first oligonucleotide comprises the sequence of SEQ ID NO: 1;
said second oligonucleotide comprises the sequence of SEQ ID NO: 7, and said first oligonucleotide comprises the sequence of SEQ ID NO: 2;
said second oligonucleotide comprises the sequence of SEQ ID NO: 10, and wherein said first oligonucleotide comprises the sequence of SEQ ID NO: 2;
said second oligonucleotide comprises the sequence of SEQ ID NO: 9, and said first oligonucleotide comprises the sequence of SEQ ID NO: 5;
said second oligonucleotide comprises the sequence of SEQ ID NO: 11, and said first oligonucleotide comprises the sequence of SEQ ID NO: 5.

11. The method according to claim 1, wherein the binding of said first oligonucleotide is determined by PCR amplification with a forward and a reverse primer.

12. The method according to claim 11, wherein said forward and reverse primer are located about 1 to 100 bp 3′ and 5′ from said first oligonucleotide.

13. The method according to claim 11, wherein said forward and reverse primer for said PCR amplification of said first oligonucleotide are selected from the group consisting of
primers comprising the sequence of SEQ ID NO: 12 and SEQ ID NO: 13, when the first oligonucleotide comprises the sequence of SEQ ID NO: 1;
primers comprising the sequence of SEQ ID NO: 14 and SEQ ID NO: 15, when the first oligonucleotide comprises the sequence of SEQ ID NO: 2;
primers comprising the sequence of SEQ ID NO: 16 and SEQ ID NO: 17, when the first oligonucleotide comprises the sequence of SEQ ID NO: 3;
primers comprising the sequence of SEQ ID NO: 18 and SEQ ID NO: 19, when the first oligonucleotide comprises the sequence of SEQ ID NO: 4;
primers comprising the sequence of SEQ ID NO: 20 and SEQ ID NO: 21, when the first oligonucleotide comprises the sequence of SEQ ID NO: 5;
primers comprising the sequence of SEQ ID NO: 22 and SEQ ID NO: 23 when the first oligonucleotide comprises the sequence of SEQ ID NO: 6;
primers comprising the sequence of SEQ ID NO: 25 and SEQ ID NO: 26 when the first oligonucleotide comprises the sequence of SEQ ID NO: 24;
or primers which have a sequence essentially identical to said primers for PCR amplification.

14. The method according to claim 1, wherein said sample is from a bird.

15. The method according to claim 14, wherein said bird is in the stage of development in which the maternal immunity of said bird disappears.

16. The method according to claim 15, wherein said bird is a duck of about 6 weeks after hatching.

17. The method according to claim 1, for determining the efficacy of a treatment against a Cp. psittaci infection.

18. A diagnostic kit for the detection of a Cp. psittaci genotype comprising one or more oligonucleotides capable of hybridizing specifically to a sequence within the DNA of a genotype of Cp. psittaci.

19. The diagnostic kit of claim 18, wherein said one or more oligonucleotides are capable of hybridizing specifically to a sequence within the ompA gene of Cp. psittaci.

20. The diagnostic kit of claim 18, wherein said one or more oligonucleotides are capable of hybridizing specifically to a sequence within the region from about nucleotide 450 to about nucleotide 600 or from about nucleotide 900 to about 1100 of the OmpA sequence corresponding to GB accession AF269281.

21. The diagnostic kit of claim 18, wherein said genotype is the EB genotype.

22. The diagnostic kit of claim 18, comprising one or more of the oligonucleotides selected from the group consisting of:
genotype-specific oligonucleotides: SEQ ID NO: 1, SEQ ID NO: 2, SEQ ID NO: 3, SEQ ID NO: 4, SEQ ID NO: 5, SEQ ID NO: 6 and 24; and
genotype-specific primers: SEQ ID NO: 7, SEQ ID NO: 8, SEQ ID NO: 9, SEQ ID NO: 10, SEQ ID NO: 11, SEQ ID NO: 12, SEQ ID NO: 13, SEQ ID NO: 14, SEQ ID NO: 15, SEQ ID NO: 16, SEQ ID NO: 17, SEQ ID NO: 18, SEQ ID NO: 19, SEQ ID NO: 20, SEQ ID NO: 21, SEQ ID NO: 22, SEQ ID NO: 23, SEQ ID NO: 25 and SEQ ID NO: 26.

23. The diagnostic kit of claim 22, comprising two oligonucleotides selected from the genotype-specific oligonucleotides SEQ ID NO: 1, SEQ ID NO: 2, SEQ ID NO: 3, SEQ ID NO: 4, SEQ ID NO: 5, SEQ ID NO: 6 and 24.

24. A strain of a Cp. psittaci bacterium, designated as Cp. psittaci genotype EB, said strain characterized in that it comprises the OmpA sequence depicted in SEQ ID NO: 51.

25. A method of generating oligonucleotide sequences useful for the discrimination between at least two genotypes of Cp. psittaci, said method comprising the steps of:
a) providing a multiple alignment of a part of the genomic sequence of said at least two Cp. psittaci genotypes,
b) identifying regions which contain sequence differences within said part of the genomic sequence,
c) synthesizing one or more oligonucleotides comprising a sequence wherein said sequences differences occur.

26. The method of claim 25, wherein said genomic sequence encodes the OmpA protein.

27. The method of claim 26, wherein said part of said genomic sequence comprises the sequence from about nucleotide 450 to about nucleotide 600 or from about nucleotide 900 to about nucleotide 1100 of the OmpA sequence corresponding to GB accession AF269281.

28. The method according to claim 25, wherein one of said at least two genotypes of Cp. psittaci is the genotype EB.
